(19) United States
(12) Patent Application Publication
(10) Pub. No.: US 2010/0152823 A1
(43) Pub. Date: Jun. 17, 2010

(54) THERAPY APPARATUS

(76) Inventor: Nicholas Muchowicz, Omaha, NE (US)

Correspondence Address: SUITER SWANTZ PC LLO, 14301 FNB PARKWAY, SUITE 220, OMAHA, NE, 68154, US

(21) Appl. No.: 12/333,005

(22) Filed: Dec. 11, 2008

(51) Int. Cl. A61F 7/00

(52) U.S. Cl. 607/112; 607/108

(57) ABSTRACT

A therapy apparatus may include: a chamber portion; a first strap portion configured for disposal about a waist of a user; and a second strap portion configured for disposal about a leg of a user.

### BACKGROUND OF THE INVENTION

Many injuries occur to human's hips, thighs and sacroiliac joints due to the numerous connective tissues and joint structures. Cryotherapy can be applied to decrease swelling and inflammation after injuries or surgeries.

This invention relates direct to application of cryotherapy to the difficult contours of the hip girdle. Due to these difficult contours of the hip, bags of ice or gel ice packs are very difficult to apply to the area. The use of bags of ice or gel ice packs may be difficult to use for many reasons. One reason is that there often nothing to secure typical forms of cryotherapy to the hip. Secondly, typical ice packs may result in unwanted moisture as ice bags melt. Similarly, gel ice packs may leak gel. Also, typical therapies may only be applied in a recumbent position.

Because of these above reasons, there is a need for a therapy apparatus that may secure itself to the body, is not messy and can be applied in multiple positions.

### SUMMARY OF THE INVENTION

### Brief Description of the Drawings

FIG. 1 depicts an isometric view of an exterior side of a therapy apparatus.

FIG. 2 depicts an isometric view of an interior of a therapy apparatus.

FIG. 3 depicts a posterior isometric view of a therapy apparatus.

FIG. 4 depicts an anterior isometric view of a therapy apparatus.

### DETAILED DESCRIPTION

The present invention provides a therapy apparatus 100 for applying cryotherapy to the hip girdle. Embodiments of the therapy apparatus 100 are shown in FIGS. 1-4. The therapy apparatus 100 may include a therapeutic wrap that can be secured to a human body. A particular implementation of the therapy apparatus 100 of FIGS. 1-4 may be used to apply cryotherapy to a hip girdle.

FIG. 1 shows an isometric view of an exterior side of a therapy apparatus 100. The therapy apparatus 100 may include an outer layer 101. The outer layer 101 may be constructed using an expandable felt material. An upper portion of the therapy apparatus 100 may include a first strap portion 102. A lower portion of the therapy apparatus 100 may include a second strap portion 103.

The first strap portion 102 and/or the second strap portion 103 of the therapy apparatus 100 may include at least one latching mechanism 104. The latching mechanism 104 may include a hook side and/or a loop side of a Velcro® fabric. The latching mechanism 104 may be operably coupled to a cooperating latch receiver 105. The latch receiver 105 may include a cooperating hook side and/or a loop side, or similar fabric which may engage the latching mechanism 104. The coupling of latching mechanism 104 the latch receiver 105 may serve to secure the first strap portion 102 around a waist 110 of a user 111.

It should be noted that numerous other latching mechanisms 104 may be employed including, but not limited to, buttons, zippers, laces and buckles, without departing from the scope and spirit of the invention.

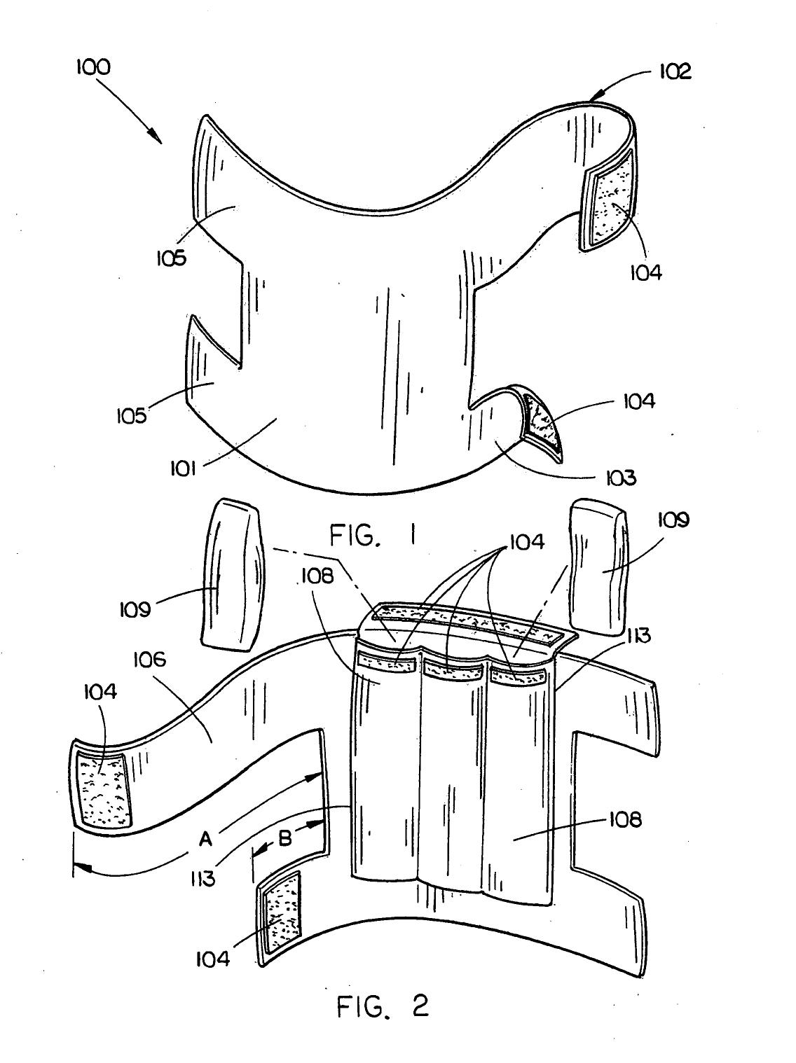

FIG. 2 shows an isometric view of an interior side of a therapy apparatus 100. The first strap portion 102 may have a transverse dimension A which is longer than a transverse dimension B of the second strap portion 103. The transverse dimension A of the first strap portion 102 may be of such length that the first strap portion 102 may be secured around a waist 110 of a user 111. The transverse dimension B of the second strap portion 103 may be of such length that it the second strap portion 103 be secured around a leg 112 of a user 111.

Such a configuration may allow the therapy apparatus 100 to closely conform to the contours of a hip girdle of a user 111 by securing the therapy apparatus 100 to both the waist 110 and leg 112 of the user 111.

The therapy apparatus 100 may include an inner layer 106. The inner layer 106 may be constructed of a porous or semi-porous material so as to provide moisture wicking from within the therapy apparatus 100 when applied to a user 111. The inner layer 106 or, cooperatively, the inner layer 106 and outer layer 101 may include at least one chamber 108. The chamber 108 may be configured to hold at least one ice pack 109. A chamber 108 may be subdivided into one or more pockets defined by stitching 113 which may operably couple the inner layer 106 and the outer layer 101. The ice pack 109 may be permanently contained within the chamber 108. Alternately, the chamber 108 may be sealed by a releasable latching mechanism 104 (e.g. a at least one piece of Velcro® fabric disposed on an inside surface of the outer layer 101 and/or at least one piece of Velcro® fabric disposed on an inside surface of the of the inner layer 106.

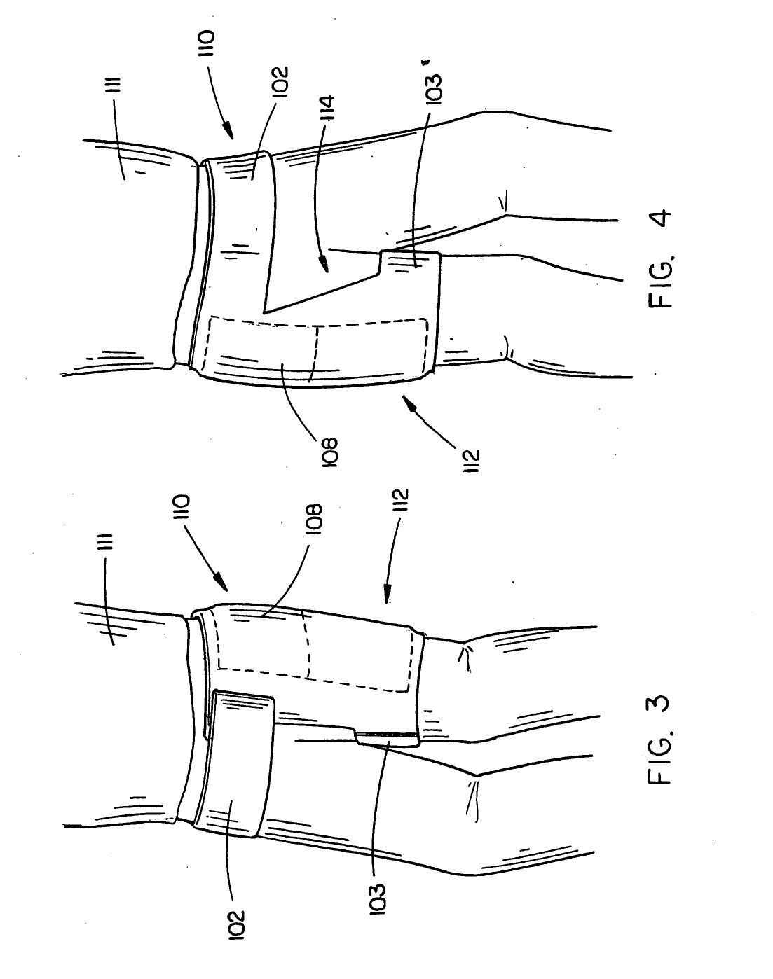

FIG. 3 shows a posterior depiction of the therapy apparatus 100 secured to a user 111. The first strap portion 102 is shown wrapping around the waist 110 of the user 111. The second strap portion 103 is shown wrapping around a leg 112 of the user 111. The coupling of latching mechanism 104 the latch receiver 105 may serve to secure the first strap portion 102 around a waist 110 of a user 111 and the second strap portion 103 around a leg 112 of a user 111. FIG. 3 provides an posterior view of the therapy apparatus 100 being applied to a right hip girdle of a user 111. In such a configuration, a chamber 108 may cover the posterior hip.

FIG. 4 illustrates an anterior view of the therapy apparatus 100 secured to a user 111. The first strap portion 102 is shown wrapping around the waist 110 of the user 111. The second strap portion 103 is shown wrapping around a leg 112 of the user 111. FIG. 4 provides an anterior view of the therapy apparatus 100 being applied to a right hip girdle of a user 111. In such a configuration, a chamber 108 may cover the anterior hip.

The therapy apparatus 100 may be constructed such that the inner layer 106 and/or outer layer 101 have a structure which follows the contours of the hip girdle (e.g. tapering 114 of the inner layer 106 and/or outer layer 101 to provide flexure points aligning with the hip socket of a user 111 so as to enhance the mobility of a user 111 wearing the therapy apparatus 100).

The materials used in constructing the therapy apparatus 100 may be of light weight so that the overall weight of the therapy apparatus 100 is insignificant to the user. Thus, the user 111 may comfortably wear the therapy apparatus 100 in any posture. For example, a user 111 may apply the therapy apparatus 100 in a sitting position, recumbent position or erect position.

While the therapy apparatus 100 has been described above with respect to cryotherapy of the lateral aspects of the hip girdle, it is fully contemplated that the therapy apparatus 100 may be configured to provide cryotherapy to various other body parts by modification of the location of the chamber 108. In an alternate configuration, the chamber 108 may be positioned more posterior, such that the therapy apparatus 100 would provide cryotherapy to the sacroiliac joint of low back In an alternate configuration, the chamber 108 may be positioned more inferiorly so as to treat thigh injuries. The various embodiments of the therapy apparatus 100 are particularly adapted as a hip wrap which allows one to treat anterior, lateral and posterior aspects of the hip girdle.

Further, while the therapy apparatus 100 has been described above with respect to cryotherapy, it is fully contemplated that the therapy apparatus 100 may be configured to provide thermal therapy (e.g. the application of hot packs), medicinal therapy (e.g. the application of medicinal compresses) and any other therapeutic component for which it may be desirable to apply to a hip girdle of a user 111.

While preferred embodiments of the invention have been described for the purpose of this disclosure, changes in the construction and arrangement of parts and the performance of steps can be made by those skilled in the art, which changes are encompassed within the spirit of this invention as defined by the appended claims.

What is claimed:

1. A therapy apparatus, wherein the therapy apparatus is configured for disposal about a waist portion and a leg portion of a user so as to operably couple the therapy apparatus to the user.

2. A therapy apparatus comprising:
a chamber portion;
a first strap portion configured for disposal about a waist of a user; and
a second strap portion configured for disposal about a leg of a user.

3. The therapy apparatus of claim 2, wherein the first strap portion is dimensioned to at least substantially approximate a circumference of a waist of a user.

4. The therapy apparatus of claim 2, wherein the second strap portion is dimensioned to at least substantially approximate a circumference of a leg of a user.

5. The therapy apparatus of claim 2, further comprising:
at least one strap portion latching mechanism.

6. The therapy apparatus of claim 2, further comprising:
at least one chamber portion latching mechanism.

7. The therapy apparatus of claim 2, further comprising:
an interior layer; and
an exterior layer.

8. The therapy apparatus of claim 7, wherein the interior layer and exterior layer define the chamber portion.

9. The therapy apparatus of claim 7, wherein at least one of the interior layer and the exterior layer comprise a tapered portion extending from the first strap portion to the second strap portion.
